(19) United States
(12) Patent Application Publication
(10) Pub. No.: US 2017/0239420 A1
(43) Pub. Date: Aug. 24, 2017

(54) Process and system employing a method to control pressures when removing, handling, and reinjecting adipose tissue

(71) Applicant: John F. Wells, Tucson, AZ (US)

(72) Inventor: John F. Wells, Tucson, AZ (US)

(21) Appl. No.: 15/132,947

(22) Filed: Apr. 19, 2016

Related U.S. Application Data
(60) Provisional application No. 62/178,756, filed on Apr. 20, 2015.
(60) Provisional application No. 62/179,885, filed on May 22, 2015.

(51) Int. Cl. A61M 5/20; A61M 1/00

(52) U.S. Cl. CPC A61M 5/2053; A61M 1/0031; A61M 2202/09; A61M 2205/3331

(57) ABSTRACT

The invention is a method and control system to manage pressure levels during reinjection procedures of viable soft tissue. Although this case specifically relates to its use in the reinjection of adipose fat and other tissue back into the body during harvesting and reinjection procedures, it can be applied to any medical procedure of introducing or re-introducing materials into the body.

### RELATED PATENTS THAT ARE CITED

| PAT. No. | Title |
|---|---|
| 8,968,272 | Closed system and method for a traumatic, |
| | low pressure, continuous harvesting, |
| | processing, and grafting of lipoaspirate |
| 8,361,042 | Fat harvesting container |
| 8,360,102 | Multi-function valve for surgical instrument |
| 8,172,832 | Fat harvesting container |
| 5,052,999 | Liposuction method and apparatus |
| 5,002,538 | Syringe adapter and method |
| 8,936,593 | Device, apparatus, and method of adipose tissue treatment |
| 8,771,592 | Device, apparatus, and method of adipose tissue treatment: |
| (B2) | Haim Epshtein, Benyamina (IL); and Boris Vaynberg, |
| | Zikron Yaakov (IL) (Reissued) |

### RELATED FOREIGN PATENT DOCUMENTS

| | | |
|---|---|---|
| 1,078,383 | February 2004 | |
| 0,743,029 | November 1993 | CA |
| 0,824,019 | July 2002 | CN |
| 2,125,986 | November 2002 | EP |
| 2,202,442 | August 1982 | EP |
| 04/299,998 | September 1988 | GB |
| 06/113,920 | October 1992 | GB |
| 11/132,843 | April 1994 | JP |
| 2003034630 | December 1999 | JP |
| WO-93/21992 | February 2003 | JP |
| WO-9909143 | November 1993 | JP |
| WO-02078644 | February 1999 | WO |
| WO-03039367 | October 2002 | WO |

### NON-PATENT REFERENCES

Liposuction, Principles and Practice Editors: Shiffman, by Melvin A., Di Giuseppe, Alberto (Eds.) Copyright 2006

Body Contouring (McGraw-Hill Plastic Surgery Atlas) Hardcover—Copyright 2010 by Michele Shermak (Author)

Body Contouring and Liposuction: Expert Consult—Online and Print, Copyright 2012 by J. Peter Rubin MD FACS (Author), and Mark L. Jewell

### CROSS REFERENCE TO RELATED PATENT APPLICATION(S)

This application is a non-provisional application being filed under 37 CFR 1.53(b) and 35 USC 111 as a divisional of the presently pending U.S. patent(s) all of which are hereby incorporated by reference.

Provisional patent application No.—62/178,756

Filing Date—Apr. 20, 2015

Title—Method and control of pressure when handling adipose tissue

### BACKGROUND OF INVENTION

A harvesting and reinjection procedure of viable soft tissue consists of several steps (U.S. Pat. No. 8,968,272) including:
1. Infiltration of tumescent fluid
2. Aspiration of viable soft tissue using liposuction
3. Processing aspirated tissue to remove debris, oil, blood, infranate, and saline
4. Reinjecting viable tissue using a cannula

Adipose tissue consists of many small adipocytes held together by fibers in larger agglomerates. During a liposuction procedure, a harvesting cannula removes these agglomerates, together with other cells and fluids, including saline solution, blood and body oil and other hydrocarbons. The harvesting cannula (U.S. Pat. No. 5,052,999) is comprised of a long, hollow metal tube (110a.) with one or more openings at or near the distal end (112), and fitted onto a hand piece (104). The cannula is inserted into a region of the body through a small incision in the skin (Khouri and Kuru U.S. Pat. No. 8,968,272). With the cannula attached to a vacuum source, adipose tissue is pulled through the openings (101) and into the inner lumen of the cannula. The adipose tissue moves through the cannula, eventually exits (105,111), and is then collected in a container.

During the liposuction procedures, viable tissues are subject to several damaging factors or forces that can compromise their viability during harvesting and reinjection procedures. These forces include:
1. Excessive negative or positive pressures
2. Shearing forces
3. Blunt forces
4. Receiving inadequate blood supply

On the vacuum side of the system, a vacuum regulator is located near the vacuum pumps. The regulator ensures that the negative pressure never exceeds the set value anywhere ‘upstream’ from the regulator. “Upstream” is defined as anywhere in the system from the distal end of the cannula (112) where the tissue and fluid enter to the regulator. During the aspiration procedure, 18 inHg of negative pressure is often recommended by experts in the medical community. But in no case should a cannula negative pressure exceed one atmosphere (29.92 inHg) which causes cell destruction and destroys tissue viability. However, previous reinjection pressures often exceeded that, and was as high as 1400 inHg positive pressure with a 1 cc syringe. Ideally, the negative pressure should only reach workable levels; which can vary from patient to patient. Workable level is defined as only as much as is needed. It has been found that pressures required to harvest adipose tissue vary from patient to patient, and can even vary from location to location in the body. This means that the surgeon will select a vacuum level, or levels, required to achieve optimum results. This invention involves a reinjection system that allows the surgeon to match and control those optimum pressures during reinjection with the pressures used during removal.

In the Prior Art, vacuum levels can currently be set and controlled during the aspiration portion of the surgical procedures, but controls are very limited and inadequate during the reinjection procedure (Khouri and Kuru U.S. Pat. No. 8,968,272). Currently, syringes are used for reinjecting the harvested and processed adipose tissue (U.S. Pat. No. 5,052,999). The Prior Art involves a variety of methods that have been used. One requires that the adipose tissue be harvested directly with the syringe connected with a cannula tip 107 (U.S. Pat. No. 5,052,999). The cannula is inserted into the patient and the plunger is pulled all the way back, creating a vacuum. Vacuum pressure cannot be regulated doing this, and the adipose tissue must fit into the small, constricted opening imposed by the luer lock. The adipose tissue is left in the large syringe to either decant slowly by gravity, or quickly in a centrifuge. Centrifuging creates enormous force and pressure on the adipocytes, drastically reducing tissue viability and potential for reuse.

Once the harvested adipose tissue has been processed and separated, it can be reinjected. The procedure of reinjection is when the harvested adipose tissue is reinjected back into areas of the body. Once the blood and any other biological materials have been removed, the processed adipose tissue is typically squeezed into a smaller syringe. A reinjection cannula (FIG. 6) (114) is attached to this smaller syringe and the tissue is reinjected back into the patient using positive pressure. The reinjection cannula has a similar design as the harvesting cannula, consisting of a hollow metal tube with one or more openings at or near the distal end, and fitted onto a hand piece. Positive pressures cannot be controlled using this method, and the agglomerates of adipose tissue are forced through the undersized luer fittings multiple times, requiring very high positive pressures in excess of 40 psi.

In the case of a clog of the system, even greater, and usually excessive, positive pressure is applied to clear the clog. A clog occurs when oversized agglomerates occlude the pathway for adipose tissue. Clogs can be hard to locate, and both time consuming and labor intensive to clear. Currently, clogs are cleared by drastically increasing the positive and/or negative pressures. When excessive positive pressure does not alleviate the clog in the system, a metal rod called a stylet is inserted into the inner lumen to dislodge the clog. Positive pressures can reach 40 psi which is approximately 80 inHg or more using a syringe. This is well in excess of the 18 inHg used during aspiration.

The reinjection procedure is performed using either a syringe (U.S. Pat. No. 5,052,999). or a peristaltic (120a.) pump. The peristaltic pumps (120a.) only have settings for flow rate and ignore positive pressure levels entirely. A standard peristaltic pump used in harvesting and reinjection can easily achieve 25 psi or more of pressure which is in excess of the 18 inHg used during aspiration. This excessive positive pressure is likely to be damaging to the viable tissue cells and can reduce cell survivability. Surgical results can be compromised and the percentage of viable cells that survive reimplantation decreases.

The present invention is a process and system to allow precise, easy to use controls over positive pressure levels during the reinjection procedure. This would allow the surgeon to limit the positive pressures so that they do not exceed negative pressure levels used in aspiration. Another benefit is to limit positive pressure so the patient does not experience any pain or discomfort. The major benefit of matching the reinjection positive pressure to the harvesting vacuum pressure is preservation of soft tissue cells such as adipocytes, thereby increasing viability and integrity of the cells: this promotes a higher percentage of the reinjected cells to permanently survive at their new destination.

### SUMMARY OF THE INVENTION

The present invention is the method and control systems for performing a harvesting and reinjection procedure where the positive pressures used in reinjection can be set to a maximum and can be controlled.

The invention may employ a number of ways and devices that can achieve this. A preferred embodiment is described in the detailed description. However, any method to achieve the sensing and adjustment of pressure is claimed within the invention.

One device utilized is a peristaltic pump with a sensor that detects pressure on the positive side of the pump. This pressure sensing can be done using many methods. Three are described below:
1. An external sensor, one that reads pressure from the outside of the tubing (702).
2. An in-line sensor, which is one that is spliced into the tubing (702).
3. Another method would be the use of sensors on or in the motor system that detect pressure through increased load on the pump. Examples of this would include a torque sensor or a current sensor to measure increased load on the pump due to increasing positive pressures in the tubing (702).

Another device claimed as inclusive the embodiment is a syringe pump. This pump can measure pressures in the system through increased resistance to the movement of the syringe plunger. The syringe pump may also use any of the three pressure sensing features described above.

Another embodiment is the use of a pressure sensor, whether in-line or external, with a regular syringe that is used by hand.

### BRIEF DESCRIPTION OF THE FIGURES

The following descriptions briefly describe the nature of each figure.

FIG. 1: Shows a system flowchart which is a representative overview of the harvesting and reinjection process.

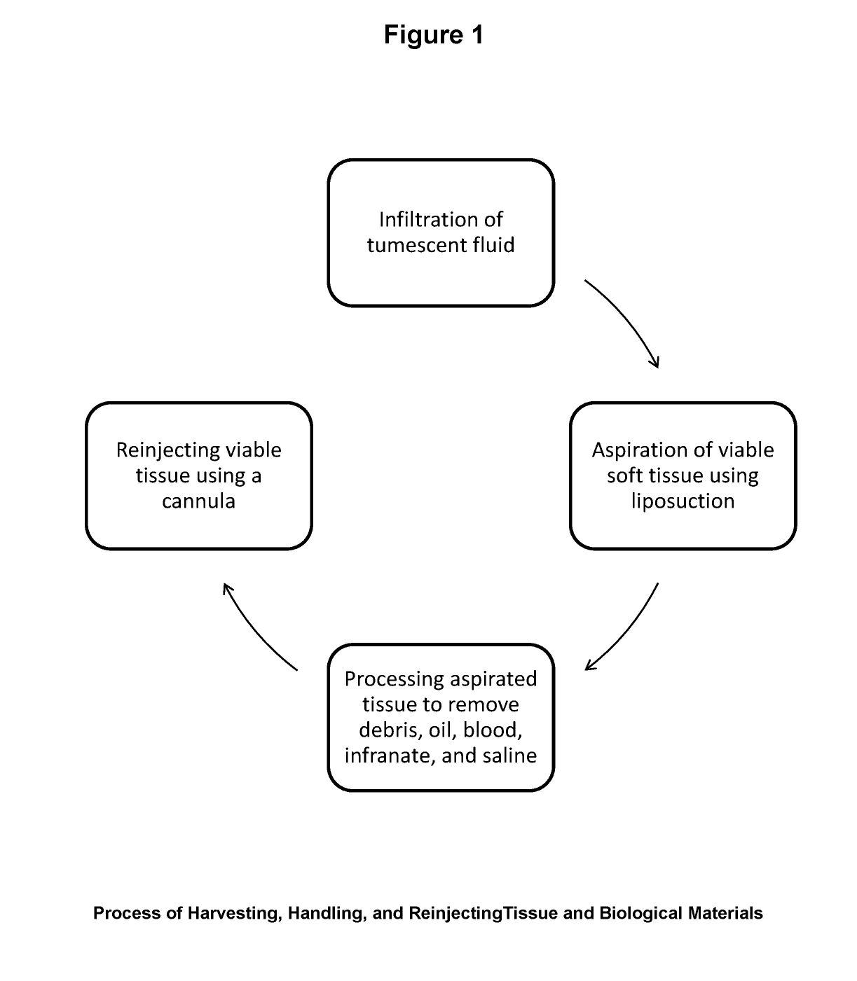

FIG. 2: Is a pictorial diagram and schematic showing the directional flow of the cannula and aspiration side of the liposuction

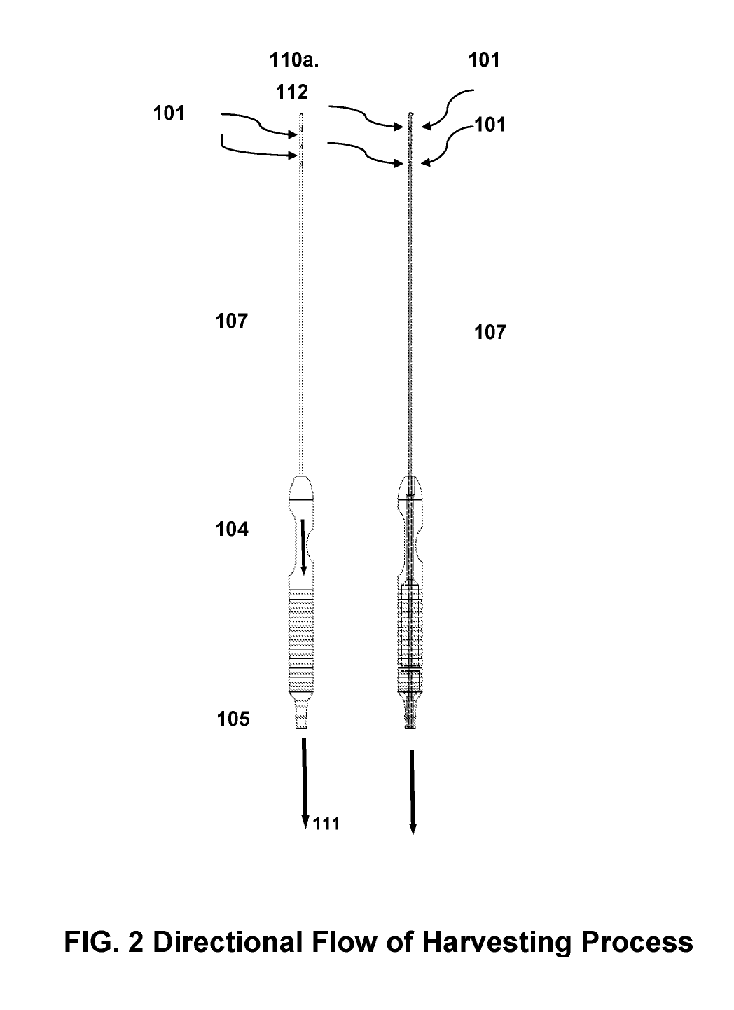

FIG. 3: Is a pictorial diagram of the peristaltic pump unit, external pressure created, and the pump head

FIG. 4: Is a cross-sectional diagram of the in-line sensor and transducer

FIG. 5: Is a schematic front view diagram of the peristaltic pump unit and pump head

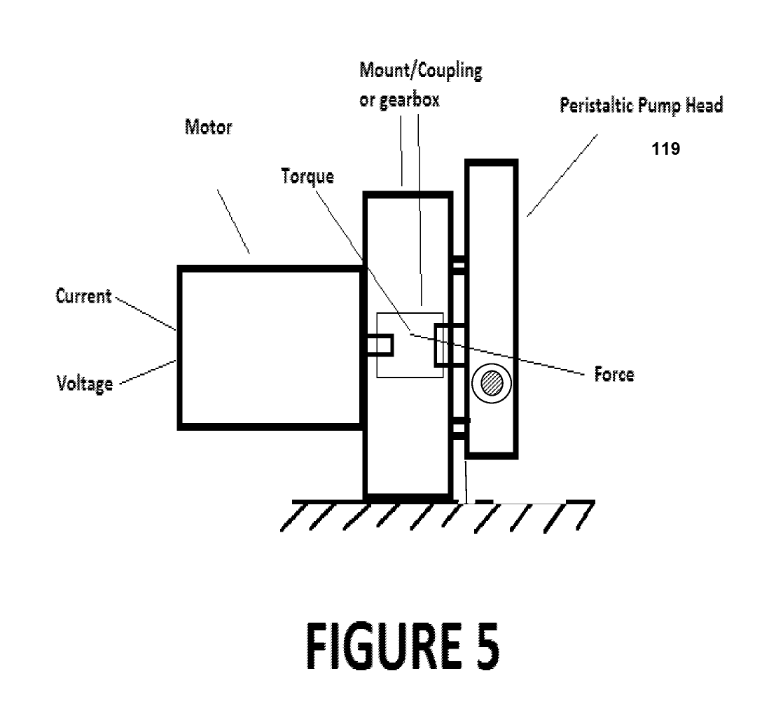

FIG. 6: Is a schematic of the reinjection cannula and the representative external flow out of the reinjection grafting cannula side of the system

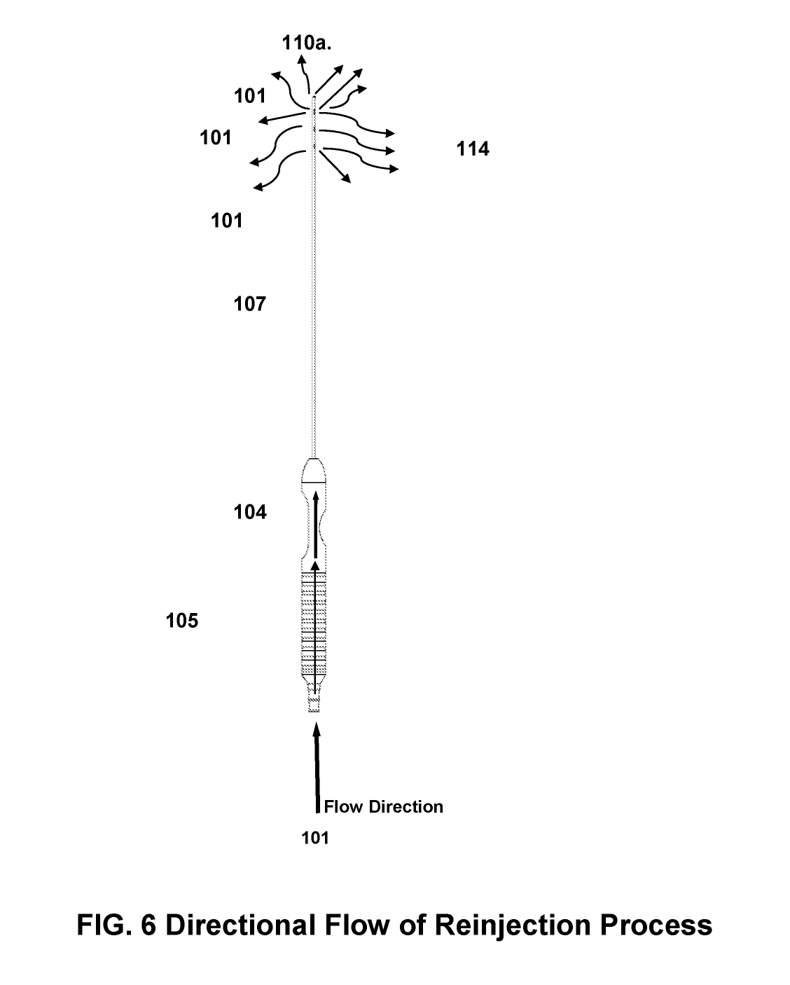

### DETAILED DESCRIPTION OF THE INVENTION

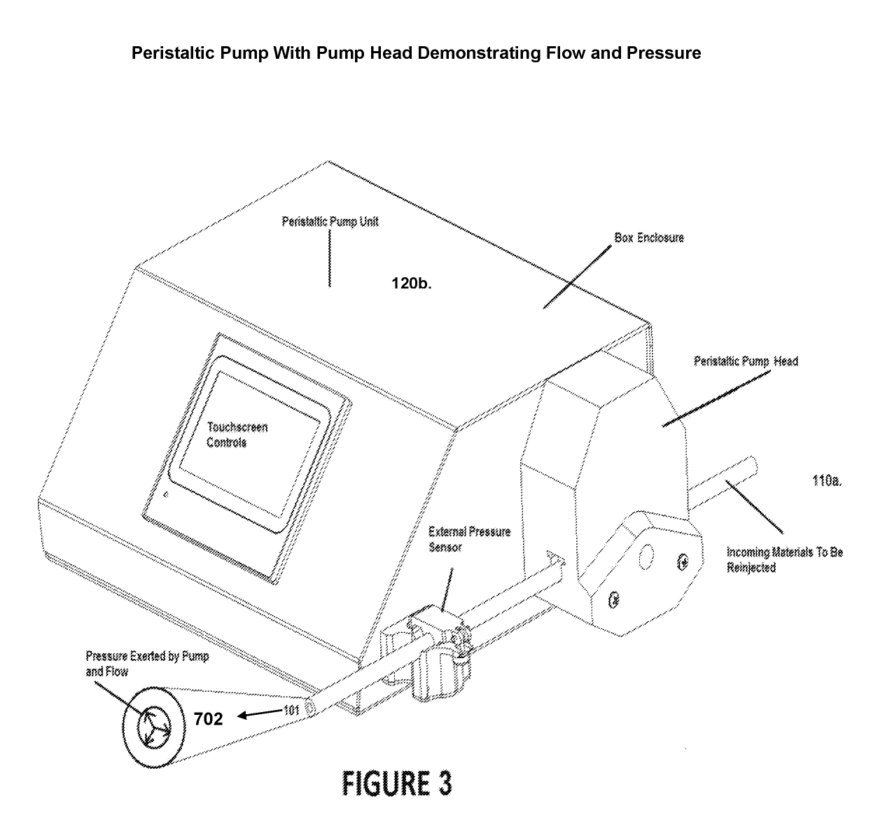

The present invention is the method and control system for performing a harvesting and reinjection procedure where the positive pressures used in reinjection are controlled. The control system consists of a reinjection pump (119), flexible tubing (702) for pumping the adipose tissue, and a means of measuring the pressure of fluid in the tubing (702). The reinjection pump uses an electric motor to drive the pump, and a user interface that allows controls over speeds and pressures (FIG. 3).

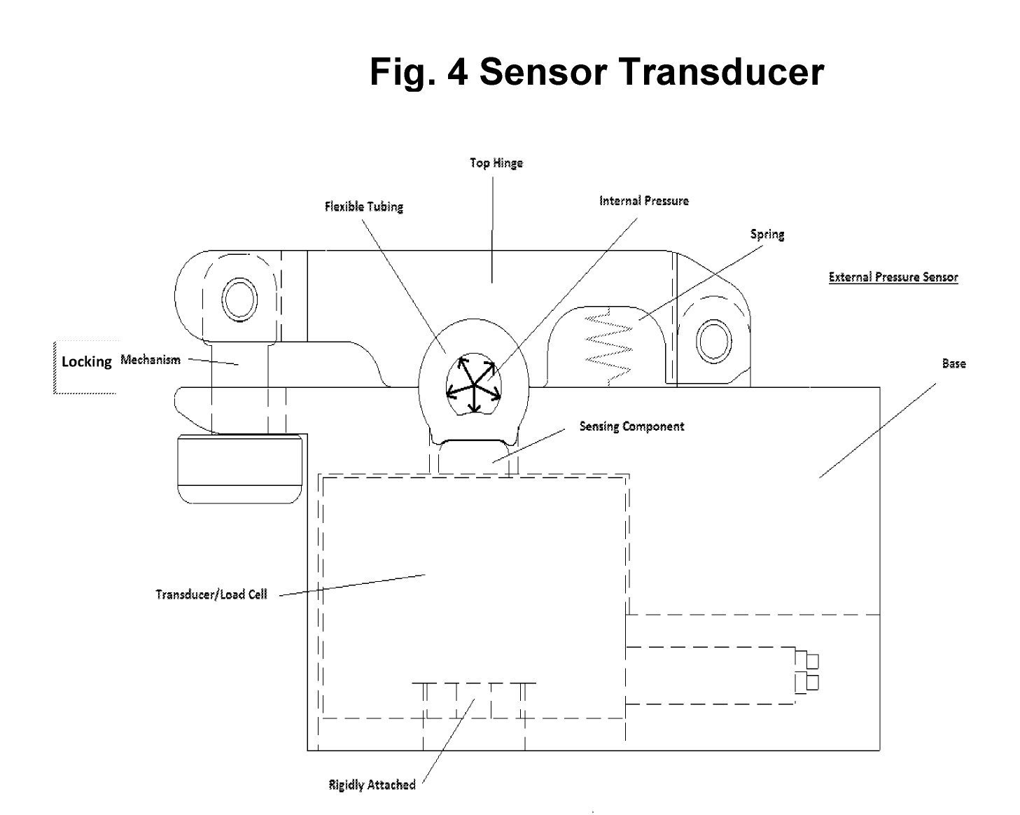

The preferred embodiment of the control system is to use a peristaltic pump (120b.) with an external sensor for measuring pressure, wherein the external sensor for pressure is a load cell (FIG. 4). A load cell is defined as “a transducer that is used to create an electrical signal whose magnitude is directly proportional to the force being measured.” (See http://en.wikipedia.org/wiki/Load_cell) The load cell (FIG. 4) is pressed and held against the flexible tubing (702), and is capable of measuring the change in pressure inside the tubing (702).

The load cell is affixed to a base component, which has a channel for the tube to sit into. A rotating hinge with a locking mechanism is attached to the base component. This hinge also has a channel for the tubing (702) to rest into. When the hinge is rotated and locked in place, the two channels line up, holding the tubing (702) in place while pushing and holding it against the sensing component of the load cell. The sensing component is described as the part, or system of parts, that transfers the force from the load to the deforming component of the load cell. The deforming component has the strain gauge on it. This sensing component is typically metal and is firmly attached to the deforming component of the load cell. This allows the load cell and strain gauge to be safely enclosed while the sensing component protrudes from the enclosure.

With the sensing component pressed and held against the flexible tubing (702), the sensor is set to a zero point. Any change in positive or negative pressure inside the tubing (702) is translated to a force pushing on the sensing component of the load cell through the wall of the flexible tube. If the hinge and base are designed correctly, it will prevent the flexible tubing (702) from expanding except in the direction of the sensing component. The internal pressure of the fluid is a force over area, so this translates to a force pushing on the surface of the sensing component. This force is directly proportional to the pressure in the tubing (702), and by using test data, a relationship between internal pressure of the tubing (702) and force on the load cell is achieved. Since the load cell is a transducer, the electrical signal is sent to and used by the reinjection pump.

Another variation of an external sensor is to use a pressure sensor. A pressure sensor is another type of transducer, like the load cell. The pressure sensor will be affixed to a base, where the base has a chamber that is filled with a medium, such as a gas or a fluid. The pressure sensor will be in fluid communication with this chamber and sealed against it. The chamber will press against the outside of the flexible tubing (702). There will be a base and hinge similar to the one described above, with channels that the flexible tubing (702) will lay into. It utilizes the concept that the flexible tubing (702) expands and contracts when pressure is in the tubing (702). If the hinge and base are properly designed, the expansion of this tubing (702) can be directed towards the chamber, which will increase the pressure of the medium in this chamber. With test data, a relationship between the pressure change in the chamber and the pressure inside the flexible tubing (702) can be made. The electrical signal created by this pressure sensor is sent to and used by the reinjection unit.

The previous description leads to an in-line sensor that measures pressure inside the tubing (702). This is achieved by having a split or extra port in the flexible tubing (702) after the pump. A pressure sensor can be attached to the end of this port, or spliced into the tubing (702) to provide pressure measurements to the reinjection pump. The setup allows the sensing component of the sensor to come in direct contact, or at least separated by a thin membrane, with the fluid being pumped through the flexible tubing (702).

Another method of sensing pressure in the flexible tubing (702) is to monitor the pump itself. As the pressure increases in the flexible tubing (702), it will put an increased load on the pump to continue to push more fluid into the tubing (702). The flexible tubing (702) expands, so the increased resistance is more gradual than if the tubing (702) was rigid. This increased resistance can be measured in a number of ways. A simple method would be to monitor the current draw and voltage of the motor. Current can be monitored only, voltage can be monitored only, or a combination of the two. With a sensitive current and/or voltage monitor and some test data, a properly sized motor

1-20. (canceled)

21. Apparatus for regulating pressure levels of a reinjected adipose tissue after harvesting of said tissue from a patient during a liposuction procedure, comprising the following:
an aspiration cannula;
a filtration device for processing said tissue after aspiration thereof;
a pump for pressurizing said tissue in a reinjection cannula; and
a pressure sensor coupled to said reinjection cannula and adapted to control a pressure level in the reinjection cannula by varying operating parameters of said pump.

22. The apparatus of claim 1, wherein said pump is a peristaltic pump and said sensor is an in-line sensor.

23. The apparatus of claim 2, wherein said in-line sensor is configured to detect a pressure increase through an increased load on the pump.
